(19) United States
(12) Patent Application Publication
(10) Pub. No.: US 2015/0299772 A1
(43) Pub. Date: Oct. 22, 2015

(54) SINGLE-STRANDED POLYNUCLEOTIDE AMPLIFICATION METHODS

(71) Applicant: Elim Biopharmaceuticals, Inc., Hayward, CA (US)

(72) Inventor: Yilin ZHANG, Hayward, CA (US)

(21) Appl. No.: 14/647,012

(22) PCT Filed: Dec. 2, 2013
(86) PCT No.: PCT/US2013/072704

§ 371 Date: May 22, 2015

Related U.S. Application Data
(60) Provisional application No. 61/732,826, filed on Dec. 3, 2012.

(51) Int. Cl. C12Q 1/68

(52) U.S. Cl. CPC C12Q 1/6806

(57) ABSTRACT

The present invention provides amplification methods for producing a population of single stranded polynucleotides from a target polynucleotide, comprising (a) extending an RNA primer in a complex comprising (i) a DNA template comprising a sequence that is complementary to the target polynucleotide, and (ii) the RNA primer, wherein the RNA primer is hybridized to the DNA template, and (b) cleaving the RNA primer with an enzyme that cleaves RNA from an RNA/DNA hybrid such that another RNA primer hybridizes to the DNA template and repeats primer extension by strand displacement.

### CROSS-REFERENCE TO RELATED APPLICATIONS

This application claims priority benefit from U.S. Provisional Patent Application No. 61/732,826 filed on Dec. 3, 2012 which is incorporated by reference in its entirety.

### TECHNICAL FIELD

This application relates generally to the fields of nucleic acid sample preparation and sequencing.

### BACKGROUND

Nucleic acid sequence analysis tools are fundamental for the identification of gene alterations, which in turn are useful for diagnosing genetic diseases, predicting responsiveness to drug treatments, and analyzing pharmacogenomics of drugs. Because sequence analyses frequently involve the determination of rare genetic alterations in a limited amount of sample, sensitivity has been a big challenge. This is particularly true when analyzing somatic mutations in a tissue sample (such as a cancer sample), which frequently contains normal cells mixed with cells harboring the mutation.

To increase sensitivity, various nucleic acid amplification methods are used. The most commonly used amplification method is polymerase chain reaction (“PCR”), which involves multiple cycles of amplifications using the Taq polymerase. Because of the inherent fidelity issues with Taq polymerases, the PCR methods frequently generate artificial mutations, which may mask the real mutations to be analyzed and make it extremely difficult to detect rare mutations in the sample. As a consequence, the accuracy of the nucleic acid methods may be compromised.

The human genomic DNA is complex and has many repetitive sequences. This presents additional challenges for sequence analyses. First, polynucleotides of interest may be significantly under-represented among the mixture of polynucleotides. Second, the cost of analyzing the complex DNA sample can be prohibitively expensive, particularly in the context of analyzing genomic DNA and detecting multiple genetic mutations. While many next generation sequencing methods have been developed, there remains a need for sensitive, accurate, and efficient methods for nucleic acid sample preparation and sequencing analyses.

All references cited herein, including patent applications and publications, are incorporated by reference in their entirety.

### SUMMARY OF THE INVENTION

The present application in one aspect provides a method of producing a population of single-stranded polynucleotides from a target polynucleotide. The first step of the method includes extending an RNA primer in a complex comprising a DNA template comprising a sequence that is complementary to the target polynucleotide and the RNA primer, wherein the RNA primer is hybridized to the DNA template. The second step of the method includes cleaving the RNA primer with an enzyme that cleaves RNA from an RNA/DNA hybrid such that another RNA primer hybridizes to the DNA template and repeats primer extension by strand replacement. The method produces a population of single-stranded polynucleotides.

The present application also provides method of analyzing a target polynucleotide. The first step of such method includes extending an RNA primer in a complex comprising a DNA template comprising a sequence that is complementary to the target polynucleotide, and the RNA primer, wherein the RNA primer is hybridized to the DNA template. The second step of the method includes cleaving the RNA primer with an enzyme that cleaves RNA from an RNA/DNA hybrid such that another RNA primer hybridizes to the DNA template and repeats primer extension by strand replacement, whereby a population of single-stranded polynucleotides is produced. In a third step includes analyzing the single-stranded polynucleotides.

In the methods above, the complex can further comprise a termination polynucleotide sequence hybridized to a region on the DNA template that is 5′ to the region the RNA primer hybridizes to. The DNA template used in the methods above can be produced from an RNA polynucleotide sequence. Optionally, the DNA template used in the methods is produced by extending an RNA primer in a complex comprising a DNA molecule and an RNA primer, wherein the RNA primer is hybridized to the DNA molecule; and cleaving the RNA primer with an enzyme that cleaves RNA from an RNA/DNA hybrid such that another RNA primer hybridizes to the DNA template and repeats primer extension by strand replacement, whereby the DNA template is produced.

In a related aspect, the present application provides a method of producing a population of single-stranded polynucleotides from a target polynucleotide sequence by incubating a reaction mixture. The reaction mixture in the method comprises a DNA template comprising a sequence that is complementary to the target polynucleotide, an RNA primer hybridizable to the DNA template, a DNA polymerase, and an enzyme that cleaves RNA from an RNA/DNA hybrid. The incubation step of the method is performed under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

The application also provides a method of analyzing a target polynucleotide by producing a population of single-stranded polynucleotides. In a first step, the population of single-stranded polynucleotides is generated by incubating a reaction mixture comprising a DNA template comprising a sequence that is complementary to the target polynucleotide, an RNA primer hybridizable to the DNA template, a DNA polymerase, and an enzyme that cleaves RNA from an RNA/DNA hybrid. In this method, the incubation is performed under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced. In a second step, the population of single-stranded polynucleotides is analyzed.

In any of the methods above, the RNA primer can be about 6 to about 20 nucleotides long. Optionally, the RNA primer can comprise a polyA sequence. Optionally, the RNA primer can comprise a random primer sequence. DNA template used in any of the methods above can optionally comprise an adaptor sequence, and the RNA primer can optionally comprise a sequence that hybridizes to the adaptor sequence. The extension step of any of the methods provided herein can be carried out by a DNA polymerase selected from a group consisting of a strand displacing DNA polymerase, a high-fidelity DNA polymerase, a polymerase that has proofreading activity, a T7 DNA polymerase, and an E. coli DNA polymerase I. The enzyme in the methods that cleaves RNA from the RNA/DNA hybrid can be RNase H. The DNA template used in the methods can optionally be a genomic DNA.

In a related aspect the application provides a kit for use in single-strand polynucleotide amplification. The kit includes an RNA primer; a DNA polymerase, and an enzyme that cleaves RNA from an RNA/DNA hybrid. The enzyme in the kit that cleaves RNA from the RNA/DNA hybrid can be RNase H. The DNA polymerase in the kit can be selected from the group consisting of selected from a group consisting of a strand displacing DNA polymerase, a high-fidelity DNA polymerase, a polymerase that has proofreading activity, a T7 DNA polymerase, and an E. coli DNA polymerase I. The RNA primer in the kit can be about 6 to about 20 nucleotide long. Optionally, RNA primer can comprise a polyA sequence. Optionally, the RNA primer comprises a random primer sequence.

### DESCRIPTION OF THE DRAWINGS

FIG. 1 depicts an exemplary method of single-strand polynucleotide amplification.

FIG. 2 depicts another exemplary method of single-strand polynucleotide amplification.

### DETAILED DESCRIPTION

The present application provides methods of single-strand polynucleotide amplification using RNA primers. The amplification methods use an RNA primer which hybridizes to a DNA template. A polymerase is used to effect the extension of the RNA primer along the DNA template. An enzyme which cleaves RNA from an RNA/DNA hybrid (such as RNase H) is then used to cleave the RNA primer from the DNA template, leaving the primer hybridization sequence on the template strand available for binding by another RNA primer and allowing initiation of another amplification cycle. Another strand is produced by the polymerase, which displaces the previously replicated strand, resulting in a displaced extension product. The repeated extensions of RNA primers lead to the production of multiple copies of single-stranded polynucleotides.

The present application therefore in one aspect provides methods of producing single-stranded polynucleotide products. In another aspect, there are provided methods of analyzing single-stranded polynucleotide products. Also provided are kits and compositions useful for methods described herein.

### I. Definitions

“Single-strand polynucleotide amplification” used herein refers to the synthesis of multiple copies of single-stranded daughter strands by repeatedly extending a single primer over a single-stranded template nucleic acid that comprises a target polynucleotide sequence. The newly synthesized nucleic acid molecules cannot serve as templates for the production of additional nucleic acid molecules during subsequent primer extension reactions.

“Amplification,” as used herein, generally refers to the process of producing two or more copies of a desired sequence.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs.

“Oligonucleotide,” as used herein, generally refers to short, generally single-stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

“Fragmenting” a polynucleotide used herein refers to breaking the polynucleotides into different polynucleotide fragments. Fragmenting can be achieved, for example, by shearing or by enzymatic reactions.

A “primer” is generally a short single-stranded polynucleotide, generally with a free 3′-OH group, that binds to a target polynucleotide of interest by hybridizing with a target sequence present on the target polynucleotide, and thereafter promotes polymerization of a polynucleotide complementary to the target polynucleotide.

The term “tag” as used herein refers to a moiety that can be used to separate a molecule to which the tag is attached to from other molecules that do not contain the tag.

The term “terminal nucleotide,” as used herein refers to the nucleotide at either the 5′ or 3′ end of a nucleic acid molecule.

“Hybridization” and “annealing” refer to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence specific manner.

An “adaptor” used herein refers to an oligonucleotide that can be joined to a polynucleotide fragment.

The term “ligation” as used herein, with respect to two polynucleotides, such as an adaptor and a polynucleotide fragment, refers to the covalent attachment of two separate polynucleotides to produce a single larger polynucleotide with a contiguous backbone.

The term “3′” generally refers to a region or position in a polynucleotide or oligonucleotide that is downstream of another region or position in the same polynucleotide or oligonucleotide.

The term “5′” generally refers to a region or position in a polynucleotide or oligonucleotide that is upstream from another region or position in the same polynucleotide or oligonucleotide.

A “5′ overhang” is a stretch of unpaired nucleotides that extend past the 5′ end of a double-stranded nucleic acid molecule. For example, a 5′ overhang can be a single unpaired nucleotide, or it can be at least 5, 10, 15 or more than 15 nucleotides long. For example, a primer can comprise, e.g., 5-25 nucleotides that are not complementary to, e.g., sequences present in a template strand and/or target polynucleotide sequence. In other words, the nucleotides of the 5′ overhang do not hybridize to the target polynucleotide sequence under conditions in which other portion(s) of the primer hybridizes to the target polynucleotide.

A “3′ overhang” is a stretch of unpaired nucleotides that extend past the 3′ end of a double-stranded nucleic acid molecule. For example, a 3′ overhang can be a single unpaired nucleotide, or it can be at least 5, 10, 15 or more than 15 nucleotides long. For example, a primer can comprise, e.g., 5-25 nucleotides that are not complementary to, e.g., sequences present in a template strand and/or target polynucleotide sequence. In other words, the nucleotides of the 3′ overhang do not hybridize to the target polynucleotide sequence under conditions in which other portion(s) of the primer hybridizes to the target polynucleotide.

The term “target polynucleotide” as used herein refers to a polynucleotide that contains one or more sequences that are of interest and under study.

An “array” used herein includes arrangement of spatially or optically addressable regions bearing nucleic acids or other molecules. When the arrays are arrays of nucleic acids, the nucleic acids may be physically adsorbed, chemically adsorbed, or covalently attached to the arrays at any point or points along the nucleic acid chain.

The term “determining,” “measuring,” “evaluating,” “assessing,” “assaying,” and “analyzing” are used interchangeably herein to refer to any form of measurement, and include determining if an element is present or not. These terms include both quantitative and/or qualitative determinations. Assessing may be relative or absolute. “assessing the presence of” includes determining the amount of something present, as well as determining whether it is present or absent.

As used herein, the term “single nucleotide polymorphism,” or “SNP” for short, refers to the alteration of a single nucleotide at a specific position in a genomic sequence, resulting in two or more alternative alleles that occur in a population at appreciable frequency (e.g., at least 1%” in a population).

The term “denaturing” as used herein refers to the separation of a nucleic acid duplex into two single-strands.

The term “enrichment” refers to the process of increasing the relative abundance of particular nucleic acid sequences in a sample relative to the level of nucleic acid sequences as a whole initially present in said sample before treatment. Thus the enrichment step provides a relative percentage or fractional increase, rather than directly increasing, for example, the absolute copy number of the nucleic acid sequences of interest. After the step of enrichment, the sample to be analyzed may be referred to as an enriched, or selected polynucleotide.

As used herein, the “complexity” of a nucleic acid sample refers to the number of different unique sequences present in that sample. A sample is considered to have “reduced complexity” if it is less complex than the nucleic acid sample from which it is derived.

As used herein, “solid support” refers to a solid or semisolid material which has the property, either inherently or through attachment of some component conferring the property (e.g., an antibody, streptavidin, nucleic acid, or other binding ligands), of binding to a tag. Such binding may be direct or indirect. Examples of solid support include, but are not limited to, nitrocellulose and nylon membranes, agarose or cellulose based beads (e.g., Sepharose) and paramagnetic beads.

As used herein, the term “library” refers to a collection of nucleic acid sequences.

As used herein, the term “hybridize specifically” means that nucleic acids hybridize with a nucleic acid of complementary sequence. As used herein, a portion of a nucleic acid molecule may hybridize specifically with a complementary sequence on another nucleic acid molecule. That is, the entire length of a nucleic acid sequence does not necessarily need to hybridize for a portion of such sequence to be “specifically hybridized” to another molecule, there may be, for example, a stretch of nucleotides at the 5′ end of a molecule that do not hybridize while a stretch at the 3′ end of the same molecule is specifically hybridized to another molecule.

A “portion” or “region,” used interchangeably herein, of a polynucleotide or oligonucleotide is a contiguous sequence of 2 or more bases. In other embodiments, a region or portion is at least about any of 3, 5, 10, 15, 20, 25 contiguous nucleotides.

Sequence “mutation,” as used herein, refers to any sequence alteration in a sequence of interest in comparison to a reference sequence. A reference sequence can be a wild type sequence or a sequence to which one wishes to compare a sequence of interest. A sequence mutation includes single nucleotide changes, or alterations of more than one nucleotide in a sequence, due to mechanisms such as substitution, deletion or insertion. Single nucleotide polymorphism (SNP) is an example of a sequence mutation as used herein.

A “complex” is a group of molecules comprising of any two or more of, e.g., a polypeptide, a nucleic acid, a primer, etc., that assemble to function together to carry out a specific reaction, e.g. a primer extension reaction. For example, in the present invention, a complex can comprise, e.g., a DNA template strand and an RNA primer that is hybridized to the DNA strand. The complex can optionally comprise a DNA polymerase that extends the RNA primer. A complex may or may not be stable and may be directly or indirectly detected. For example, as is described herein, given certain components of a reaction, and the type of product(s) of the reaction, existence of a complex can be inferred. For purposes of this invention, a complex is generally an intermediate with respect to formation the final amplification product(s), i.e., daughter strands.

As used herein, “cleaving” or “to cleave” refers to enzymatic digestion, e.g., of the RNA portion of an RNA: DNA hybrid.

A nucleic acid or primer is “complementary” to another nucleic acid when at least two contiguous bases of, e.g., a first nucleic acid or a primer, can combine in an antiparallel association or hybridize with at least a subsequence of a second nucleic acid to form a duplex. In some embodiments, complementarity between e.g., a primer and a target polynucleotide sequence, is not 100% perfect.

A “primer extension reaction” refers to a molecular reaction in which a nucleic acid polymerase adds one or more nucleotides to the 3′ terminus of a primer that is hybridized to a target polynucleotide sequence in a template-specific manner, i.e., wherein the daughter strand produced by the primer extension reaction is complementary to the target polynucleotide sequence. Extension not only refers to the first nucleotide added to the 3′ terminus of a primer, but also includes any further extension of a polynucleotide formed by the extended primer.

A “random primer” as used herein, is a primer that comprises a sequence that is based on a statistical expectation (or an empirical observation) that the sequence of the random primer is hybridizable (under a given set of conditions) to one or more sequences a nucleic acid sample, e.g., a genomic DNA, a population of RNAs, etc. The sequence of a random primer may or may not be naturally-occurring, or may or may not be present in a pool of sequences in a sample of interest. The amplification of a plurality of different daughter strands in a single reaction mixture would generally, but not necessarily, employ a multiplicity, preferably a large multiplicity, of random primers. As is well understood in the art, a “random primer” can also refer to a primer that is a member of a population of primers (a plurality of random primers) which collectively are designed to hybridize to a desired and/or a significant number of target sequences. A random primer may hybridize at a plurality of sites on a template nucleic acid. The use of random primers provides a method for generating primer extension products complementary to a target polynucleotide which does not require prior knowledge of the exact sequence of the target.

A “reaction mixture” is an assemblage of components (e.g., one or more polypeptides, nucleic acids, and/or primers), which, under suitable conditions, react to carry out a specific reaction, e.g. a primer extension reaction.

A “termination polynucleotide sequence” or a “termination sequence”, as used interchangeably herein, is a polynucleotide sequence which promotes the termination of a primer extension reaction by diverting or blocking further extension of the daughter strand beyond a specified position on the target polynucleotide sequence. A termination sequence comprises a portion (or region) that generally hybridizes to the target polynucleotide sequence at a location 3′ to the primer hybridization site. The portion of termination sequence capable of hybridizing to the target polynucleotide sequence may or may not encompass the entire termination sequence. For example, a termination sequence can be, e.g., an oligonucleotide that binds, generally with high affinity, to the template nucleic acid at a location 5′ to the termination site and 3′ to the primer hybridization site. Its 3′ end may or may not be blocked for extension by DNA polymerase. The site, point or region of the target polynucleotide that is last replicated by the DNA polymerase before the termination of a primer extension reaction is a “termination site” or “termination point”.

It is understood that aspect and embodiments of the invention described herein include “consisting” and/or “consisting essentially of” aspects and embodiments.

As used herein, the singular form “a”, “an”, and “the” includes plural references unless indicated otherwise.

As is understood by one skilled in the art, reference to “about” a value or parameter herein includes (and describes) embodiments that are directed to that value or parameter per se. For example, description referring to “about X” includes description of “X”.

### II. Methods of Single-Strand Polynucleotide Amplification

The methods described herein generally involve use of an RNA primer, and are generally carried out in an in vitro context, e.g., in a reaction chamber or a container suitable for the reactions. The various steps of the methods in some embodiments are carried out within the same reaction container. Alternatively, one or more steps described herein are carried out in separate reaction containers. In some embodiments, the method is carried out at room temperature. In some embodiments, the method is carried out at a constant temperature. In some embodiments, the method is carried out at a temperature that is below 37° C. In some embodiments, the method is carried out at a temperature that is above 37° C.

The single-strand polynucleotide amplification methods generally work as follows: an RNA primer is allowed to hybridize (i.e., anneal) to the DNA template (for example one strand of a double-stranded DNA or a DNA strand that has been converted from a single-stranded RNA by reverse transcription). A polymerase (such as DNA polymerase) is used to effect the extension of the RNA primer using the DNA template. An enzyme which cleaves RNA from an RNA/DNA hybrid (such as RNase H) cleaves (removes) RNA sequence from the hybrid, leaving sequence on the template strand available for binding by another RNA primer. Another strand is produced by the polymerase (such as DNA polymerase), which displaces the previously replicated strand, resulting in a displaced extension product. The single-strand polynucleotides described herein can be generated from single-stranded or double-stranded DNA or RNA.

In some embodiments, the template DNA is obtained from genomic DNA, DNA produced by primer extension reaction, cDNA, mitochondrial DNA, chloroplast DNA, plasmid DNA, bacterial artificial chromosomes, yeast artificial chromosomes, or a combination thereof. In some embodiments, the template DNA is present in a sample. In some embodiments, the sample is a tissue sample. In some embodiments, the sample is a body fluid sample. In some embodiments, the sample is a tumor sample. In some embodiments, the sample is obtained from an individual having cancer. In some embodiments, the sample is polynucleotides extracted from a sample (such as a tissue sample). In some embodiments, the sample is a single cell. In some embodiments, the sample is polynucleotides extracted from a single cell.

In some embodiments, the present application provides a method of producing a population of single-stranded polynucleotides from a target polynucleotide, comprising: (a) extending an RNA primer in a complex comprising: (i) a DNA template comprising a sequence that is complementary to the target polynucleotide, and (ii) the RNA primer, wherein the RNA primer is hybridized to the DNA template; and (b) cleaving the RNA primer with an enzyme that cleaves RNA from an RNA/DNA hybrid such that another RNA primer hybridizes to the DNA template and repeats primer extension by strand replacement, whereby a population of single-stranded polynucleotides are produced.

In some embodiments, there is provided a method of producing a population of single-stranded polynucleotides from a target polynucleotide, comprising: (a) annealing an RNA primer to a DNA template comprising a sequence that is complementary to the target polynucleotide, (b) extending the RNA primer by primer extension reaction; (c) cleaving the RNA primer with an enzyme that cleaves RNA from an RNA/DNA hybrid such that another RNA primer hybridizes to the DNA template and repeats primer extension by strand replacement, whereby a population of single-stranded polynucleotides are produced. In some embodiments, the template DNA is obtained from genomic DNA, DNA produced by primer extension reaction, cDNA, mitochondrial DNA, chloroplast DNA, plasmid DNA, bacterial artificial chromosomes, yeast artificial chromosomes, or a combination thereof. In some embodiments, the template DNA is present in a sample. In some embodiments, the sample is a tissue sample. In some embodiments, the sample is a body fluid sample. In some embodiments, the sample is a tumor tissue sample. In some embodiments, the sample is obtained from an individual having cancer. In some embodiments, the sample is polynucleotides extracted from a sample (such as a tissue sample for example a tumor tissue sample). In some embodiments, the sample is a single cell. In some embodiments, the sample is polynucleotides extracted from a single cell.

In some embodiments, there is provided a method of producing a population of single-stranded polynucleotides from a double-stranded DNA in a sample, comprising: (a) denaturing the double-stranded DNA; (a) annealing an RNA primer to one strand of the double-stranded DNA, (b) allowing the RNA primer to extension by primer extension reaction; (c) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced. In some embodiments, the double-stranded DNA is genomic DNA. In some embodiments, the double-stranded DNA (such as genomic DNA) is in a tissue sample (for example a tumor tissue sample). In some embodiments, the double-stranded DNA (such as genomic DNA) is extracted from a tissue sample (for example a tumor tissue sample). In some embodiments, the double-stranded DNA (such as genomic DNA) is extracted from a body fluid sample. In some embodiments, the double-stranded DNA (such as genomic DNA) is extracted from the body fluid sample. In some embodiments, the double-stranded DNA (such as genomic DNA) is in a single cell. In some embodiments, the double-stranded DNA (such as genomic DNA) is extracted from a single cell.

In some embodiments, there is provide a method of producing a population of single-stranded polynucleotides from a single-stranded RNA in a sample, comprising: (a) reverse transcribing the single-stranded RNA into single-stranded DNA template, (b) annealing an RNA primer to the DNA template, (c) extending the RNA primer by primer extension reaction; (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced. In some embodiments, the single-stranded RNA is mRNA. In some embodiments, the single-stranded RNA (such as mRNA) is in a tissue sample (for example a tumor tissue sample). In some embodiments, the single-stranded RNA (such as mRNA) is extracted from a tissue sample (for example a tumor tissue sample). In some embodiments, the sample is a body fluid. In some embodiments, the single-stranded RNA (such as mRNA) is extracted from the body fluid sample. In some embodiments, the single-stranded RNA (such as mRNA) is in a single cell. In some embodiments, the single-stranded RNA (such as mRNA) is extracted from a single cell.

In some embodiments, there is provided a method of analyzing (for example sequencing) a target polynucleotide, comprising: (a) extending an RNA primer in a complex comprising: (i) a DNA template comprising a sequence that is complementary to the target polynucleotide, and (ii) the RNA primer, wherein the RNA primer is hybridized to the DNA template; (b) cleaving the RNA primer with an enzyme that cleaves RNA from an RNA/DNA hybrid such that another RNA primer hybridizes to the DNA template and repeats primer extension by strand replacement, whereby a population of single-stranded polynucleotides are produced; and (c) analyzing (for example sequencing) the single-stranded polynucleotides.

In some embodiments, there is provided a method of analyzing (for example sequencing) a double-stranded DNA in a sample, comprising: (a) denaturing the double-stranded DNA, (b) annealing an RNA primer to one strand of the double-stranded DNA, (c) extending the RNA primer by primer extension reaction; (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced; and (e) analyzing (for example sequencing) the single-stranded polynucleotides. In some embodiments, the double-stranded DNA is genomic DNA. In some embodiments, the double-stranded DNA (such as genomic DNA) is in a tissue sample (for example a tumor tissue sample). In some embodiments, the double-stranded DNA (such as genomic DNA) is extracted from a tissue sample (for example a tumor tissue sample). In some embodiments, the double-stranded DNA (such as genomic DNA) is extracted from a body fluid sample. In some embodiments, the double-stranded DNA (such as genomic DNA) is extracted from the body fluid sample. In some embodiments, the double-stranded DNA (such as genomic DNA) is in a single cell. In some embodiments, the double-stranded DNA (such as genomic DNA) is extracted from a single cell.

In some embodiments, there is provided a method of analyzing (for example sequencing) a single-stranded RNA in a sample, comprising: (a) reverse transcribing the single-stranded RNA into single-stranded DNA template, (b) annealing an RNA primer to the DNA template, (c) extending the RNA primer by primer extension reaction; (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced; and (e) analyzing (for example sequencing) the single-stranded polynucleotides. In some embodiments, the single-stranded RNA is mRNA. In some embodiments, the single-stranded RNA (such as mRNA) is in a tissue sample (for example a tumor tissue sample). In some embodiments, the single-stranded RNA (such as mRNA) is extracted from a tissue sample (for example a tumor tissue sample). In some embodiments, the sample is a body fluid. In some embodiments, the single-stranded RNA (such as mRNA) is extracted from the body fluid sample. In some embodiments, the single-stranded RNA (such as mRNA) is in a single cell. In some embodiments, the single-stranded RNA (such as mRNA) is extracted from a single cell.

In some embodiments, there is provided a method of analyzing (for example sequencing) a target polynucleotide by analyzing a population of single-stranded polynucleotides generated from the target polynucleotide, wherein the population of single-stranded polynucleotides are generated by a method comprising: (a) extending an RNA primer in a complex comprising: (i) a DNA template comprising a sequence that is complementary to the target polynucleotide, and (ii) the RNA primer, wherein the RNA primer is hybridized to the DNA template; (b) cleaving the RNA primer with an enzyme that cleaves RNA from an RNA/DNA hybrid such that another RNA primer hybridizes to the DNA template and repeats primer extension by strand replacement, whereby a population of single-stranded polynucleotides are produced; and (c) analyzing (for example sequencing) the single-stranded polynucleotides.

In some embodiments, there is provided a method of analyzing (for example sequencing) double-stranded DNA by analyzing a population of single-stranded polynucleotides generated from the target polynucleotide, wherein the population of single-stranded polynucleotides are generated by a method comprising: (a) denaturing the double-stranded DNA, (b) annealing an RNA primer to one strand of the double-stranded DNA, (c) extending the RNA primer to by primer extension reaction; and (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced. In some embodiments, the double-stranded DNA is genomic DNA. In some embodiments, the double-stranded DNA (such as genomic DNA) is in a tissue sample (for example a tumor tissue sample). In some embodiments, the double-stranded DNA (such as genomic DNA) is extracted from a tissue sample (for example a tumor tissue sample). In some embodiments, the double-stranded DNA (such as genomic DNA) is in a body fluid sample. In some embodiments, the double-stranded DNA (such as genomic DNA) is extracted from the body fluid sample. In some embodiments, the double-stranded DNA (such as genomic DNA) is in a single cell. In some embodiments, the double-stranded DNA (such as genomic DNA) is extracted from a single cell.

In some embodiments, there is provided a method of analyzing (for example sequencing) single-stranded RNA by analyzing a population of single-stranded polynucleotides generated from the target polynucleotide, wherein the population of single-stranded polynucleotides are generated by a method comprising: (a) reverse transcribing the single-stranded RNA into single-stranded DNA template, (b) annealing an RNA primer to the DNA template, (c) extending the RNA primer by a primer extension reaction; (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced. In some embodiments, the single-stranded RNA (such as mRNA) is in a tissue sample (for example a tumor tissue sample). In some embodiments, the single-stranded RNA (such as mRNA) is extracted from a tissue sample (for example a tumor tissue sample). In some embodiments, the sample is a body fluid. In some embodiments, the single-stranded RNA (such as mRNA) is extracted from the body fluid sample. In some embodiments, the single-stranded RNA (such as mRNA) is in a single cell. In some embodiments, the single-stranded RNA (such as mRNA) is extracted from a single cell.

In some embodiments, there is provided a method of detecting the presence or absence of a single-stranded RNA comprising a sequence of interest in a sample comprising RNA, comprising: a) reverse transcribing the RNA into single-stranded DNA templates; b) annealing an RNA primer to the DNA template; c) extending the RNA primer by primer extension reaction; d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced, and e) analyzing the population of single-stranded polynucleotides to determine the presence or absence of a single-stranded RNA comprising a sequence of interest in the sample. In some embodiments, the single-stranded RNA (such as mRNA) is in a tissue sample (for example a tumor tissue sample). In some embodiments, the single-stranded RNA (such as mRNA) is extracted from a tissue sample (for example a tumor tissue sample). In some embodiments, the sample is a body fluid. In some embodiments, the single-stranded RNA (such as mRNA) is extracted from the body fluid sample. In some embodiments, the single-stranded RNA (such as mRNA) is in a single cell. In some embodiments, the single-stranded RNA (such as mRNA) is extracted from a single cell.

In some embodiments, there is provided a method of detecting a mutation in a double-stranded DNA (such as genomic DNA), comprising: (a) denaturing the double-stranded DNA, (b) annealing an RNA primer to one strand of the double-stranded DNA, (c) extending the RNA primer to by primer extension reaction; and (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced, and (e) analyzing the single-stranded polynucleotides to detect the mutation. In some embodiments, the double-stranded DNA is genomic DNA. In some embodiments, the double-stranded DNA (such as genomic DNA) is in a tissue sample (for example a tumor tissue sample). In some embodiments, the double-stranded DNA (such as genomic DNA) is extracted from a tissue sample (for example a tumor tissue sample). In some embodiments, the double-stranded DNA (such as genomic DNA) is extracted from a body fluid sample. In some embodiments, the double-stranded DNA (such as genomic DNA) is extracted from the body fluid sample. In some embodiments, the double-stranded DNA (such as genomic DNA) is in a single cell. In some embodiments, the double-stranded DNA (such as genomic DNA) is extracted from a single cell.

In some embodiments, there is provided a method of producing a population of single-stranded polynucleotides from a target polynucleotide sequence comprising incubating a reaction mixture, the reaction mixture comprising: (a) a DNA template comprising a sequence that is complementary to the target polynucleotide; (b) an RNA primer hybridizable to the DNA template, (c) a DNA polymerase, and (d) an enzyme that cleaves RNA from an RNA/DNA hybrid, wherein the incubation is under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

In some embodiments, there is provided a method of analyzing a target polynucleotide; comprising: 1) producing a population of single-stranded polynucleotides are generated by a method comprising incubating a reaction mixture comprising: (a) a DNA template comprising a sequence that is complementary to the target polynucleotide; (b) an RNA primer hybridizable to the DNA template, (c) a DNA polymerase, and (d) an enzyme that cleaves RNA from an RNA/DNA hybrid, wherein the incubation is under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced; and 2) analyzing the single-stranded polynucleotides.

In some embodiments, there is provided a method of analyzing a target polynucleotide by analyzing a population of single-stranded polynucleotides produced from the target polynucleotide, wherein the population of single-stranded polynucleotides are generated by a method comprising incubating a reaction mixture comprising: (a) a DNA template comprising a sequence that is complementary to the target polynucleotide; (b) an RNA primer hybridizable to the DNA template, (c) a DNA polymerase, and (d) an enzyme that cleaves RNA from an RNA/DNA hybrid, wherein the incubation is under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

In some embodiments, there is provided a method of producing a population of single-stranded polynucleotides from a double-stranded DNA (such as genomic DNA) in a sample, comprising: a) denaturing the double-stranded DNA in the sample; b) adding a RNA primer hybridizable to one strand of the double-stranded DNA, a DNA polymerase, and an enzyme that cleaves RNA from an RNA/DNA hybrid to the sample to create a reaction mixture, and c) incubating the reaction mixture under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

In some embodiments, there is provided a method of producing a population of single-stranded polynucleotides from a double-stranded DNA (such as genomic DNA) in a sample, comprising: a) denaturing the double-stranded DNA in the sample; b) adding a RNA primer hybridizable to one strand of the double-stranded DNA, a DNA polymerase, and an enzyme that cleaves RNA from an RNA/DNA hybrid to the sample to create a reaction mixture, and c) incubating the reaction mixture under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

In some embodiments, there is provided a method of analyzing double-stranded DNA in a sample; comprising: 1) producing a population of single-stranded polynucleotides are generated by a method comprising incubating a reaction mixture comprising: a) denaturing the double-stranded DNA in the sample; b) adding a RNA primer hybridizable to one strand of the double-stranded DNA, a DNA polymerase, and an enzyme that cleaves RNA from an RNA/DNA hybrid to the sample to create a reaction mixture, and c) incubating the reaction mixture under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced; and 2) analyzing the single-stranded polynucleotides.

In some embodiments, there is provided a method of analyzing a double-stranded DNA by analyzing a population of single-stranded polynucleotides produced from the target polynucleotide, wherein the population of single-stranded polynucleotides are generated by a method comprising: a) denaturing the double-stranded DNA in the sample; b) adding a RNA primer hybridizable to one strand of the double-stranded DNA, a DNA polymerase, and an enzyme that cleaves RNA from an RNA/DNA hybrid to the sample to create a reaction mixture, and c) incubating the reaction mixture under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

In some embodiments, there is provided a method of producing a population of single-stranded polynucleotides from a single-stranded RNA (such as mRNA) in a sample, comprising: a) reverse transcribing the single-stranded RNA into a single-stranded DNA template; b) adding a RNA primer hybridizable to the DNA template, a DNA polymerase, and an enzyme that cleaves RNA from an RNA/DNA hybrid to the sample to create a reaction mixture, and c) incubating the reaction mixture under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

In some embodiments, there is provided a method of analyzing a single-stranded RNA (such as mRNA) in a sample, comprising: a) reverse transcribing the single-stranded RNA into a single-stranded DNA template; b) adding a RNA primer hybridizable to the DNA template, a DNA polymerase, and an enzyme that cleaves RNA from an RNA/DNA hybrid to the sample to create a reaction mixture, and c) incubating the reaction mixture under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced; and 2) analyzing the single-stranded polynucleotides.

In some embodiments, there is provided a method of analyzing a single-stranded RNA (such as mRNA) in a sample by analyzing a population of single-stranded polynucleotides produced from the target polynucleotide, wherein the population of single-stranded polynucleotides are generated by a method a) reverse transcribing the single-stranded RNA into a single-stranded DNA template; b) adding a RNA primer hybridizable to the DNA template, a DNA polymerase, and an enzyme that cleaves RNA from an RNA/DNA hybrid to the sample to create a reaction mixture, and c) incubating the reaction mixture under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

The reaction mixture described herein can further comprise other components, such as one or more components in the reaction medium described herein.

The total length of the RNA primer can be from about 10 to about 40 nucleotides, including for example about 15 to about 30 nucleotides, about 20 to about 25 nucleotides. In some embodiments, the length of the primer is at least about any of 10, 15, 20, 25, 30, 35, and 40 nucleotides. In some embodiments, the length of the primer is no more than about any of 15, 20, 25, 30, 40, or 50 nucleotides. To achieve hybridization (which, as is well known and understood in the art, depends on other factors such as, for example, ionic strength and temperature), the primers in some embodiments are at least about 60%, 70%, 75%, 80%, 85%, 90%, or 95% complementary to a portion of the DNA template.

In some embodiments, the RNA primer comprises a sequence that is complementary to a sequence of interest. In some embodiments, the RNA primer is a random primer (such as N9). In some embodiments, the RNA primer is complementary to a sequence on an adaptor added to the DNA template (either though limited round of amplification using primers comprising adaptor sequences or through ligation).

The amplification methods described herein in some embodiments use a DNA polymerase. In some embodiments, the DNA polymerase is one that is capable of extending a nucleic acid primer along a nucleic acid template that is comprised at least predominantly of deoxyribonucleotides. The polymerase should be able to displace a nucleic acid strand from the polynucleotide to which the displaced strand is bound, and, generally, the more strand displacement capability the polymerase exhibits (i.e., compared to other polymerases which do not have as much strand displacement capability) is preferable. In some embodiments, the DNA polymerase has high affinity for binding at the 3′-end of an oligonucleotide hybridized to a nucleic acid strand. In some embodiments, the DNA polymerase does not possess substantial nicking activity. In some embodiments, the polymerase has little or no 5′→3′ exonuclease activity so as to minimize degradation of primer or primer extension polynucleotides. Generally, this exonuclease activity is dependent on factors such as pH, salt concentration, and so forth, all of which are familiar to one skilled in the art. Mutant DNA polymerases in which the 5′→3′ exonuclease activity has been deleted, are known in the art and are suitable for the amplification methods described herein. Suitable DNA polymerases for use in the methods and compositions of the present invention include those disclosed in U.S. Pat. Nos. 5,648,211 and 5,744,312, which include exo-Vent (New England Biolabs), exo-Deep Vent (New England Biolabs), Bst (BioRad), exo-Pfu (Stratagene), Bca (Panvera), sequencing grade Taq (Promega), and thermostable DNA polymerases from thermoanaerobacter thermohydrosulfuricus. In some embodiments, the DNA polymerase displaces primer extension products from the template nucleic acid in at least about 25%, more preferably at least about 50%, even more preferably at least about 75%, and most preferably at least about 90%, of the incidence of contact between the polymerase and the 5′ end of the primer extension product. In some embodiments, the use of thermostable DNA polymerases with strand displacement activity is used. Such polymerases are known in the art, such as described in U.S. Pat. No. 5,744,312 (and references cited therein). Preferably, the DNA polymerase has little to no proofreading activity. In some embodiments, the DNA polymerase is selected from the group consisting of a strand-displacing DNA polymerase, a high fidelity DNA polymerase, a polymerase that has proofreading activity, a T7 DNA polymerase, and an E. coli DNA polymerase I.

The enzyme that cleaves RNA from an RNA/DNA hybrid in some embodiments is a ribonuclease that cleaves ribonucleotides regardless of the identity and type of nucleotides adjacent to the ribonucleotide to be cleaved. In some embodiments, the enzyme cleaves independent of sequence identity. Examples of suitable ribonucleases for the methods and compositions of the present invention are well known in the art, including ribonuclease H (RNase H).

Appropriate reaction media and conditions for carrying out the methods described herein are those that permit nucleic acid amplification. Such media and conditions are known to persons of skill in the art, and are described in various publications, such as U.S. Pat. No. 5,679,512 and PCT Pub. No. WO99/42618. For example, a buffer may be Tris buffer, although other buffers can also be used as long as the buffer components are non-inhibitory to enzyme components of the methods of the invention. The pH can be about 5 to about 11, for example from about 6 to about 10, from about 7 to about 9, from about 7.5 to about 8.5, or about 8.5. The reaction medium can also include bivalent metal ions, such as Mg²⁺ or Mn²⁺, at a final concentration of free ions that is within the range of from about 0.01 to about 10 mM, including for example from about 1 to about 5 mM. The reaction medium can also include other salts, such as KCl, that contribute to the total ionic strength of the medium. For example, the range of a salt such as KCl is from about 0 to about 100 mM, including from about 0 to about 75 mM, such as from about 0 to about 50 mM. The reaction mixture may also contain a single-stranded DNA binding protein; for example, it may contain 3 ug T4gp32 (USB). The reaction medium can further include additives that could affect performance of the amplification reactions, but that are not integral to the activity of the enzyme components of the methods. Such additives include proteins such as BSA, and non-ionic detergents such as NP40 or Triton. Reagents, such as DTT, that are capable of maintaining enzyme activities can also be included; for example, DTT may be included at a concentration of about 1 to about 5 mM. Such reagents are known in the art.

Where appropriate, an RNase inhibitor (such as Rnasine) that does not inhibit the activity of the RNase employed in the method can also be included. The reaction can occur at a constant temperature or at varying temperatures. In some embodiments, the reactions are performed isothermally, which avoids the cumbersome thermocycling process. The amplification reaction is carried out at a temperature that permits hybridization of the RNA primer (or terminating sequence) of the present invention to the template polynucleotide and that does not substantially inhibit the activity of the enzymes employed. The temperature can be in the range of about 25° C. to about 85° C., including for example about 30° C. to about 75° C., about 37° C. to about 70° C., or about 55° C. In some embodiments, the reaction is carried out at a temperature in the range of about 25° C. to about 85° C., about 30° C. to about 75° C., and about 37° C. to about 70° C.

The reaction mixture containing the primers, probes, and samples may first be denatured (for example by incubation at 95° C. for about 2 to about 5 min), and the primer(s) allowed to anneal to target (for example at 55° C. for about 5 min).

Nucleotide and/or nucleotide analogs, such as deoxyribonucleoside triphosphates, that can be employed for synthesis of the primer extension products in the methods of the invention can be provided in the amount of from about 50 to about 2500 μM, about 100 to about 2000 μM, about 500 to about 1700 μM, or about 800 to about 1500 μM. Deoxyribose nucleoside triphosphates (dNTPs) may be used at a concentration of, for example, about 250 to about 500 uM. In some embodiments, a nucleotide or nucleotide analog whose presence in the primer extension strand enhances displacement of the strand (for example, by causing base pairing that is weaker than conventional AT, CG base pairing) is included. Such nucleotide or nucleotide analogs include deoxyinosine and other modified bases, all of which are known in the art. Nucleotides and/or analogs, such as ribonucleoside triphosphates, that can be employed for synthesis of the RNA transcripts in the methods of the invention are provided in the amount of from about 0.25 to about 6 mM, about 0.5 to about 5 mM, about 0.75 to about 4 mM, or about 1 to about 3 mM.

The oligonucleotide components of the amplification reactions of the invention are generally in excess of the number of target nucleic acid sequence to be amplified. They can be provided at about or at least about any of the following: 10, 10², 10⁴, 10⁶, 10⁸, 10¹⁰, 10¹² times the amount of target nucleic acid. The RNA primer can be provided at about or at least about any of the following concentrations: 50 nM, 100 nM, 500 nM, 1000 nM, 2500 nM, or 5000 nM.

In one embodiment, the foregoing components are added simultaneously at the initiation of the amplification process. In another embodiment, components are added in any order prior to or after appropriate time points during the amplification process, as required and/or permitted by the amplification reaction. Such time points can be readily identified by a person of skill in the art. The enzymes used for nucleic acid amplification according to the methods of the present invention can be added to the reaction mixture either prior to the nucleic acid denaturation step, following the denaturation step, or following hybridization of the primer to the target DNA, as determined by their thermal stability and/or other considerations known to the person of skill in the art.

The amplification reactions can be stopped at various time points, and resumed at a later time. Said time points can be readily identified by a person of skill in the art. Methods for stopping the reactions are known in the art, including, for example, cooling the reaction mixture to a temperature that inhibits enzyme activity. Methods for resuming the reactions are also known in the art, including, for example, raising the temperature of the reaction mixture to a temperature that permits enzyme activity. In some embodiments, one or more of the components of the reactions is replenished prior to, at, or following the resumption of the reactions. Alternatively, the reaction can be allowed to proceed (i.e., from start to finish) without interruption.

In some embodiments, a termination sequence is used in conjunction with a RNA primer in the amplification reaction. The termination sequence comprises a sequence that hybridizes to a region on the DNA template that is downstream of the region where the RNA primer is hybridized to, which serves as a blocking point for the extension reaction.

When needed, bar code sequences can be added to the polynucleotides generated by the methods described herein, for example by subjecting the single-stranded polynucleotides to limited number of cycles (for example 2, 3, 4, 5, 6, 7, 8, 9, 10, 11, 12, 13, 14, or 15 cycles) of PCR reactions using primers comprising the bar code sequences. In some embodiments, bar code sequences are added by ligating one or more adaptors having the bar code sequences to one or both ends of the polynucleotides prior to, simultaneously, or after the single-strand polynucleotide amplification methods. Adaptors not containing the bar code sequences are also contemplated. Provisional Application entitled “Compositions and Methods of Nucleic Acid Preparation and Analysis,” filed concurrently with and incorporated herein by reference, describes various uses of adaptors.

In some embodiments, the polynucleotides to be amplified/analyzed (such as double-stranded target DNA or single-stranded RNA) are present in the sample at an amount of less than about 500 ng. In some embodiments, each sample comprises at least about 1 pg, 10 pg, 100 pg, 1 ng, 10 ng, 20 ng, 30 ng, 40 ng, 50 ng, 60 ng, 75 ng, 100 ng, 150 ng, 200 ng, 250 ng, 300 ng, 400 ng, 500 ng, 1 μg, 1.5 μg, 2 μg, or more polynucleotide material. In some embodiments, the sample comprises less than about 1 pg, 10 pg, 100 pg, 1 ng, 10 ng, 20 ng, 30 ng, 40 ng, 50 ng, 60 ng, 75 ng, 100 ng, 150 ng, 200 ng, 250 ng, 300 ng, 400 ng, 500 ng, 1 ∞g, 1.5 μg, or 2 μg polynucleotide material.

In some embodiments, the sample is processed, for example by subjecting the DNA in the sample to denaturation and/or fragmentation prior to the methods described herein. DNA fragmentation can be carried out in a many different ways. For example, the polynucleotides (such as double-stranded DNA) can be fragmented by acoustic sonication, and/or treatment with one or more enzymes under conditions suitable for the one or more enzymes to generate random double-stranded nucleic acid breaks (which can include DNase I, Fragmentase, and variants thereof). In some embodiments, the fragmentation comprises treating the double-stranded target DNA with one or more restriction endonucleases. The fragments generated can have an average length of about 50 to about 10,000 nucleotides, such as an average length of about 100 to about 10,000 nucleotides, or about 500 to about 25,000 nucleotides.

Also provided are methods of making polynucleotide libraries using any one of the amplification methods described herein. Such libraries can be useful, for example, for next generation sequencing. For example, in some embodiments, there is provided a method of producing a library of polynucleotides, wherein the method comprises generating a population of single-stranded polynucleotides from a target polynucleotide by: (a) extending an RNA primer in a complex comprising: (i) a DNA template comprising a sequence that is complementary to the target polynucleotide, and (ii) the RNA primer, wherein the RNA primer is hybridized to the DNA template; and (b) cleaving the RNA primer with an enzyme that cleaves RNA from an RNA/DNA hybrid such that another RNA primer hybridizes to the DNA template and repeats primer extension by strand replacement, whereby a population of single-stranded polynucleotides are produced.

In some embodiments, there is provided a method of producing a library of polynucleotides, wherein the method comprise generating a population of single-stranded polynucleotides from a double-stranded DNA (such as genomic DNA) by: (a) denaturing the double-stranded DNA; (a) annealing an RNA primer to one strand of the double-stranded DNA, (b) allowing the RNA primer to extension by primer extension reaction; (c) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced.

In some embodiments, there is provide a method of producing a library of polynucleotides, wherein the method comprise generating a population of single-stranded polynucleotides from a single-stranded RNA (such as mRNA) by: (a) reverse transcribing the single-stranded RNA into single-stranded DNA template, (b) annealing an RNA primer to the DNA template, (c) extending the RNA primer by primer extension reaction; (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced.

In some embodiments, there are provided methods of making polynucleotides microarrays using any one of the amplification methods described herein. The microarrays can be used, for example, for next generation sequencing, mutation analysis, expression profiling, diagnosing diseases, and the like. For example, in some embodiments, there is provided a method of producing a polynucleotide microarrays, wherein the method comprises 1) generating a population of single-stranded polynucleotides from a target polynucleotide by: (a) extending an RNA primer in a complex comprising: (i) a DNA template comprising a sequence that is complementary to the target polynucleotide, and (ii) the RNA primer, wherein the RNA primer is hybridized to the DNA template; and (b) cleaving the RNA primer with an enzyme that cleaves RNA from an RNA/DNA hybrid such that another RNA primer hybridizes to the DNA template and repeats primer extension by strand replacement, whereby a population of single-stranded polynucleotides are produced, and 2) attaching the single-stranded polynucleotides to a solid support.

In some embodiments, there is provided a method of producing a polynucleotide microarray, wherein the method comprises: 1) generating a population of single-stranded polynucleotides from a double-stranded DNA by: (a) denaturing the double-stranded DNA; (a) annealing an RNA primer to one strand of the double-stranded DNA, (b) allowing the RNA primer to extension by primer extension reaction; (c) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced; and 2) attaching the single-stranded polynucleotides to a solid support.

In some embodiments, there is provided a method of producing a polynucleotide microarray, wherein the method comprises: 1) generating a population of single-stranded polynucleotides from a single-stranded RNA by: (a) reverse transcribing the single-stranded RNA into single-stranded DNA template, (b) annealing an RNA primer to the DNA template, (c) extending the RNA primer by primer extension reaction; (d) cleaving the RNA primer with an enzyme that cleaves RNA from an RNA/DNA hybrid such that another RNA primer hybridizes to the same DNA strand and repeats primer extension by strand replacement, whereby a population of single-stranded polynucleotides are produced; and 2) attaching the single-stranded polynucleotides to a solid support.

### III. Methods of Analyzing Polynucleotides

The methods described herein in some embodiments comprise analysis of polynucleotides. The analyses can include, but are not limited to, polynucleotide sequencing, mutation analysis, determination of polymorphism, etc. The methods described herein are particularly useful for identifying mutations in a polynucleotide sample, predicting responsiveness of an individual to a drug; predicting pharmacokinetics of drug in an individual, predicting therapeutic outcome of a treatment in an individual. The methods can also be useful for genetic testing such as genetic testing for prenatal screening.

The polynucleotides can be analyzed by any analysis methods, including, but not limited to, DNA sequencing (using Sanger, pyrosequencing or the sequencing systems of Roche/454, Helicos, Illumina/Solexa, and ABI (SOLID)), a polymerase chain reaction assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, or a sandwich hybridization assay, for example. The polynucleotide molecules can be sequenced or analyzed for the presence of SNPs or other differences relative to a reference sequence.

In some embodiments, the polynucleotides generated by the methods described herein can be used for NP haplotyping of a chromosomal region that contains two or more SNPS, for enriching for DNA sequences for paired-end sequencing methods, for generating target fragments for long-read sequences, isolating inversion, deletion, and translocation breakpoints, for sequencing entire gene regions (exons and introns) to uncover mutations causing aberrant splicing or regulation, and for the production of long probes for chromosome imaging, e.g., Bionanomatrix, optical mapping, or fiber-FISH-based methods.

Polymorphisms, particularly single nucleotide polymorphism (“SNP”) are essentially randomly distributed throughout the genome. A polymorphism may be an insertion, deletion, duplication, or rearrangement of any length of a sequence, including single nucleotide deletions, insertions, or base change. The polymorphism may be naturally occurring, or it may be associated with variant phenotypes. The use of the methods described herein, for example through the enrichment of the sequences of interest, allows substantially reproducible access to substantially similar reduced-complexity subpopulations in different individuals in a population or even in different samples from a single individual. Because polymorphisms are essentially randomly distributed throughout the genome, a number of polymorphic sequences will be present in the reduced-complexity population of nucleic acid sequences. Such reduced-complexity subpopulation can be analyzed to either identify polymorphisms or to determine the genotype of polymorphic loci within that sub-population.

The methods described herein can also be useful, for example, in the field of pharmacogenomics, which seeks to correlate the knowledge of specific alleles of polymorphic loci with the way in which individuals in a population respond to particular drug. A broad estimate is that, for every drug, between 10% and 40% of individuals do not respond optimally. In order to create a response profile for a given drug, the genotype with regard to polymorphic loci of those individuals receiving the drug must be correlated with the therapeutic outcome of the drug. This is frequently performed with analysis of a large number of polymorphic loci. Once a genetic drug response profile has been estimated by analysis of polymorphic loci in a population, a clinical patient's genotype with respect to those loci related to responses to particular drugs must be determined. Therefore, the ability to identify the sequence of a large number of polymorphic loci in a large number of individuals is important for both establishment of a drug response profile and for identification of an individual's genotype for clinical applications.

The polynucleotides generated using the methods described herein can be subjected to sequencing analysis using the Illumina sequencing method. The Illumina sequencing method includes bridge amplification technology, in which primers bound to a solid phase are used in the extension and amplification of solution phase single-stranded nucleic acid acids prior to SBS. (See, e.g., Mercier, et al. (2005) “Solid Phase DNA Amplification: A Brownian Dynamics Study of Crowding Effects.” Biophysical Journal 89: 32-42; Bing, et al. (1996) “Bridge Amplification: A Solid Phase PCR System for the Amplification and Detection of Allelic Differences in Single Copy Genes.” Proceedings of the Seventh International Symposium on Human Identification, Promega Corporation Madison, Wis.)

Illumina sequencing technology entails preparing single-stranded nucleic acids flanked with paired-end adapter sequences. Each of the paired-end adapters contains a unique primer hybridization sequence. The nucleic acids are distributed on to a flow cell surface that is coated with single-stranded oligonucleotides that correspond to the primer hybridization sequences present on the adapters flanking the single-stranded nucleic acids. The single-stranded, adapter-ligated nucleic acids are bound to the surface of the flow cell and exposed to reagents for polymerase-based extension. Priming occurs as the free/distal end of a ligated fragment “bridges” to a complementary oligonucleotide on the surface, and during the annealing step, the extension product from one bound primer forms a second bridge strand to the other bound primer. Repeated denaturation and extension results in localized amplification of single molecules in millions of unique locations, creating clonal “clusters” across the flow cell surface.

The flow cell is then placed in a fluidics cassette within a sequencing module, where primers, DNA polymerase, and fluorescently-labeled, reversibly terminated nucleotides, e.g., A, C, G, and T, are added to permit the incorporation of a single nucleotide into each clonal DNA in each cluster. Each incorporation step is followed by the high-resolution imaging of the entire flow cell to identify the nucleotides that were incorporated at each cluster location on the flow cell. After the imaging step, a chemical step is performed to deblock the 3′ ends of the incorporated nucleotides to permit the subsequent incorporation of another nucleotide. Iterative cycles are performed to generate a series of images each representing a single base extension at a specific cluster. This system typically produces sequence reads of up to 20-50 nucleotides. Further details regarding this sequencing system are discussed in, e.g., Bennett, et al. (2005) “Toward the 1,000 dollars human genome.” Pharmacogenomics 6: 373-382; Bennett, S. (2004) “Solexa Ltd.” Pharmacogenomics 5: 433-438; and Bentley, D. R. (2006) “Whole genome re-sequencing.” Curr Opin Genet Dev 16: 545-52.

The first stage in preparing template for the Illumina system is DNA fragmentation by nebulization. However, the wide size distribution of generated fragments is uneconomical, as the 20-200 fragments that can be used in subsequent template preparation steps represent approximately 10% of the total DNA after nebulization. Moreover, approximately half of the DNA vaporizes after nebulization, meaning that only 5% of the original DNA is used to prepare sequencing template. Additionally, 50% of the DNA strands in the clonal clusters that are formed during bridge amplification, as strands with free 5′ ends are removed prior to the sequencing reaction.

In some embodiments, the polynucleotides generated by the methods described herein are analyzed using single-molecule real-time sequencing. Single molecule real-time sequencing (SMRT) is another massively parallel sequencing technology that can be used to sequence circularized single-stranded nucleic acids in a high-throughput manner. Developed and commercialized by Pacific Biosciences, SMRT technology relies on arrays of multiplexed zero-mode waveguides (ZMWs) in which, e.g., thousands of sequencing reactions can take place simultaneously. The ZMW is a structure that creates an illuminated observation volume that is small enough to observe, e.g., the template-dependent synthesis of a single-stranded DNA molecule by a single DNA polymerase (See, e.g., Levene, et al. (2003) “Zero Mode Waveguides for Single Molecule Analysis at High Concentrations,” Science 299: 682-686). When a DNA polymerase incorporates complementary, fluorescently labeled nucleotides into the DNA strand that is being synthesized, the enzyme holds each nucleotide within the detection volume for tens of milliseconds, e.g., orders of magnitude longer than the amount of time it takes an unincorporated nucleotide to diffuse in and out of the detection volume. During this time, the fluorophore emits fluorescent light whose color corresponds to the nucleotide base's identity. Then, as part of the nucleotide incorporation cycle, the polymerase cleaves the bond that previously held the fluorophore in place and the dye diffuses out of the detection volume. Following incorporation, the signal immediately returns to baseline and the process repeats. Additional descriptions of ZMWs and their application in single molecule analyses, such as SMRT sequencing can be found in, e.g., Published U.S. Patent Application No. 2003/0044781, and U.S. Pat. No. 6,917,726, each of which is incorporated herein by reference in its entirety for all purposes. See also, Levene et al. (2003) “Zero Mode Waveguides for single Molecule Analysis at High Concentrations,” Science 299:682-686 and Eid, et al. (2009) “Real-Time DNA Sequencing from Single Polymerase Molecules.” Science 323:133-138.

The polynucleotides generated by the methods described herein can be adapted for use with the SMRT sequencing platform. For example, following synthesis, the single-stranded polynucleotides can be circularized using an enzyme that catalyzes the intramolecular ligation of single-stranded DNA fragments, e.g., CircLigase™, CircLigase™ II, or ThermoPhage™, and distributed to ZMWs. Alternatively, the daughter strands can be fragmented prior to circularization. Optionally, sequences of interest can be enriched from a population of fragmented daughter strands, e.g., as described below, prior to circularization.

The single-stranded polynucleotides produced by the methods described herein can be further enriched for polynucleotides of interest prior to the analysis of the polynucleotides of interest. The enrichment generally involves contacting a population of single-stranded polynucleotides with a set of probes, wherein the probes hybridize to one or more polymucleotides of interest, thereby enriching polynucleotides of interest. The enrichment methods described herein reduce the complexity of the polynucleotide sequences to be analyzed and allow the polynucleotides of interest to be better represented in the pool.

Thus, in some embodiments, the method further comprises an enrichment step comprising: 1) contacting a population of single-stranded polynucleotides generated by any of the methods described herein with a set of probes that are hybridizable to one or more regions on the target polynucleotides; and 2) separating polynucleotides that are bound to the probes from the rest of the polynucleotides, wherein polynucleotides comprising the one or more desired regions are enriched.

The probes used herein can be hybridizable to any regions of interest. In some embodiments, the one or more desired regions are regions where oncogenes are located. In some embodiments, the one or more desired regions are regions where a mutation of interest is located. In some embodiments, the one or more desired regions are regions where a polymorphism is located.

The number of probes may be selected based on the complexity level of the sample material and the sequence length desired to be sequenced. The methods described herein may be done using a single oligonucleotide or a plurality (i.e., a mixture of at least 2, at least 5, at least 10, at least 50, at least 100, at least 500, at least 1000, at least 10,000, at least 100,000, or more) of different oligonucleotides. These oligonucleotides can be used to enrich for a plurality (i.e., at least 2, at least 5, at least 10, at least 50, at least 100, at least 500, at least 1000, at least 10,000, at least 100,000, or more) different regions on the polynucleotide sequence.

The probes used in the methods described herein can be of any length, including, but not limited to, about 200 to about 500, about 500 to about 1,000, about 1,000 to about 2,000, about 2,000 to about 5,000, about 5,000 to about 10,000, about 10,000 to about 20,000 nucleotides long. The probes in some embodiments are provided in access to the polynucleotides to be enriched. For example, in some embodiments, the probes are at least about any of 10, 102, 103, 104, or more times the amount of the polynucleotides to be enriched. In some embodiments, the probes are no more than about 10, 102, 103, or 104 times the amount of the polynucleotides to be enriched.

The level of complexity reduction obtained by the enrichment method may enable reduction of 10%, 20%, 30%, 40%, 50%, 60%, 70%, 80%, 90%, 95%, or 99% of the complexity of the initial polynucleotide pool, or may involve selection of only a few percent of the polynucleotides, or even a few thousand base pairs. For example, when the initial polynucleotide pool is generated from a genomic DNA, the complexity of the polynucleotides may be reduced from 3 billion base pairs to 10 million base pairs or less, depending on the size of the initial genome and the level of reduction required. Using this method, highly repetitive DNA sequences which comprise, for example 40% of the human genomic DNA, can be removed quickly and efficiently from a complex population.

### IV. Kits, Compositions, Reagents, and Article of Manufacture

Also provided herein are kits, reagents, and articles of manufacture useful for the methods described herein.

In some embodiments, there is provided a kit useful for any one of the methods described herein. In some embodiments, the kit comprises an RNA primer. In some embodiments, the kit further comprises one or more of: 1) a DNA polymerase (such as a DNA-dependent DNA polymerase and/or an RNA-dependent DNA polymerase), 2) a DNA endonuclease, 3) a DNA kinase, 4) a DNA exonuclease, 5) a DNA endonuclease, 6) an enzyme comprising RNaseH activity, and 7) one or more buffers suitable for one or more of the elements contained in the kit.

In some embodiments, the kit comprises an enzyme that cleaves RNA from an RNA/DNA hybrid, including but not limited to, RNase H or RNase I. In some embodiments, the kit further comprises a DNA polymerase, such as a DNA polymerase selected from the group consisting of a strand displacing DNA polymerase, a high-fidelity DNA polymerase, a polymerase that has proofreading activity, a T7 DNA polymerase, and an E. coli DNA polymerase. In some embodiments, the kit comprises a DNA ligase. In some embodiments, the kit comprises buffer suitable for any one of the reactions described herein, i.e., ligation, single-strand polynucleotide amplification, and enrichment, etc. These components may be provided in a separate kit, or provided together with the adaptors and primers described herein. In some embodiments, the kit comprises one or more components in the reaction medium described herein.

The kits described herein may further comprise instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kits or components thereof (i.e., associated with the packaging or subpackaging) etc. In some embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

The various components of the kit may be in separate containers, where the containers may be contained within a single housing, e.g., a box.

Further provided herein are methods of making any of the articles of manufacture described herein.

### EXAMPLES

The following are examples of methods and compositions of the invention. It is understood that various other embodiments may be practiced, given the general description provided above.

### Example 1

### Amplifying Single-Stranded Polynucleotides from a Double-Stranded DNA

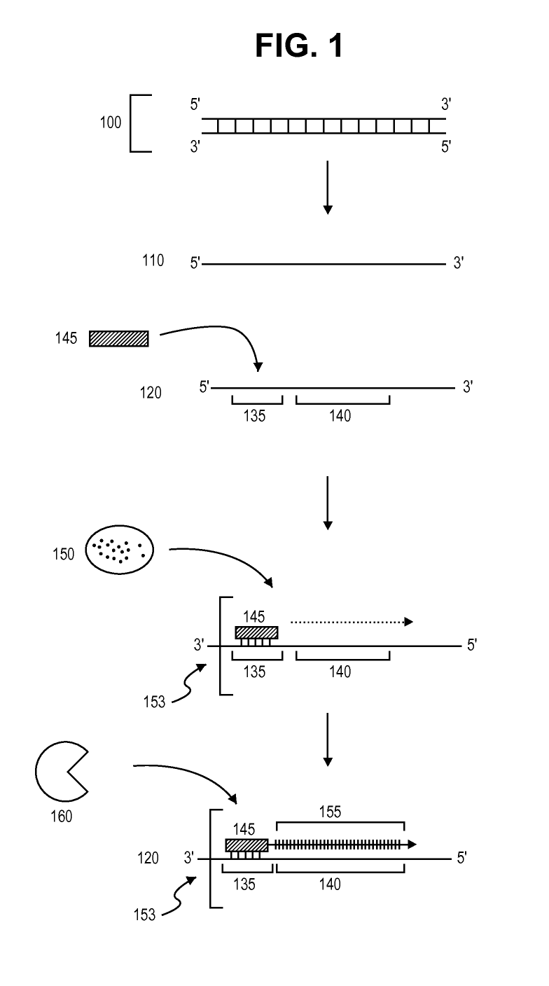

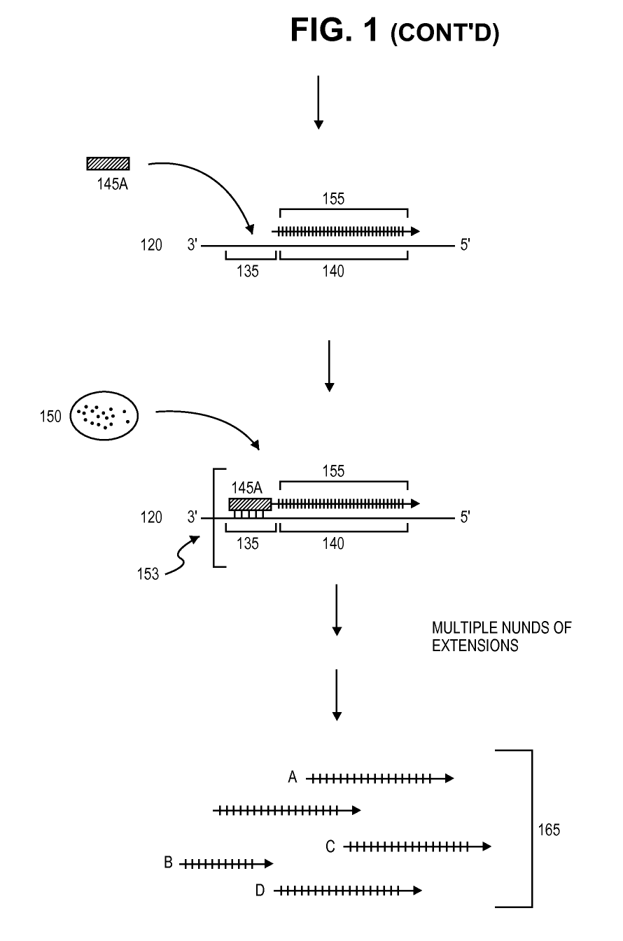

This example provides one exemplary method of single-strand polynucleotide amplification. The steps of this method are schematically depicted in FIG. 1.

In a first step, double-stranded DNA 100 is provided. Double-stranded DNA 100 can be obtained from any source described herein using methods known in the art. Double-stranded DNA 100 is then denatured (for example by incubation at 95° C. for about 2 to about 5 min) to produce single DNA strands 110 and 120. Single DNA strand 120 comprises primer annealing site 135 and template sequence 140.

Next, RNA primer 145 hybridizes to primer annealing site 135 on single DNA strand 120 to form RNA/DNA hybrid 153. RNA primer 145 is then extended via the sequential addition of nucleotides in a template-specific manner by DNA polymerase 150 to produce target polynucleotide 155. The RNA portion RNA/DNA hybrid 153 is then cleaved (removed) by enzyme 160, which specifically digests RNA that is hybridized to DNA. The digestion of the RNA portion of RNA/DNA hybrid 153 by enzyme 160 exposes primer annealing site 135, permitting RNA primer 145A to hybridize to single DNA strand 120. Primer 145A is then be extended by polymerase 150, displacing target polynucleotide 155 while producing another target polynucleotide.

Repeated cycles of RNA primer annealing, primer extension, and primer digestion produces population of single-stranded polynucleotides, which is represented in FIG. 1 as population 165.

### Example 2

### Amplifying Single-Stranded Polynucleotides from an RNA

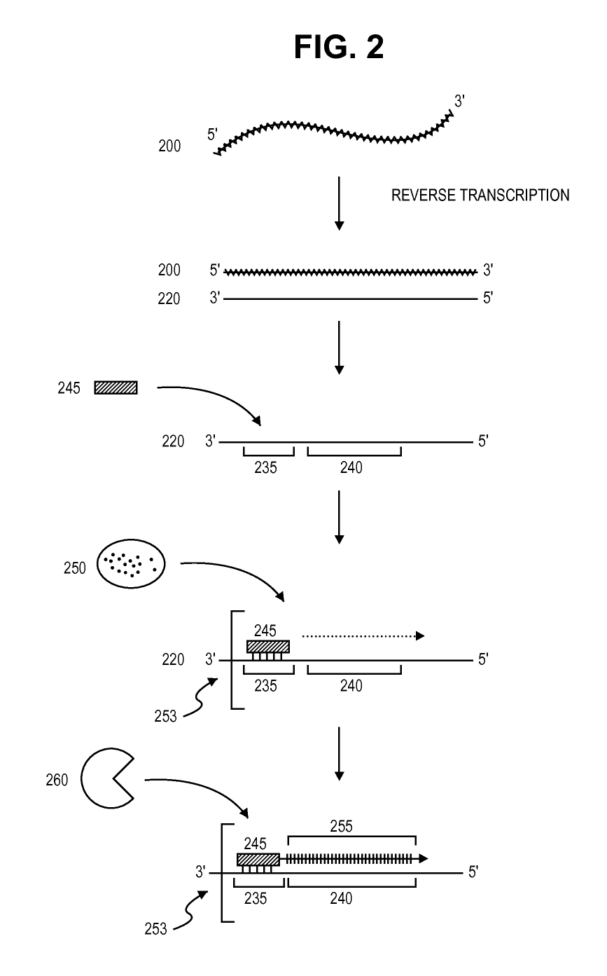

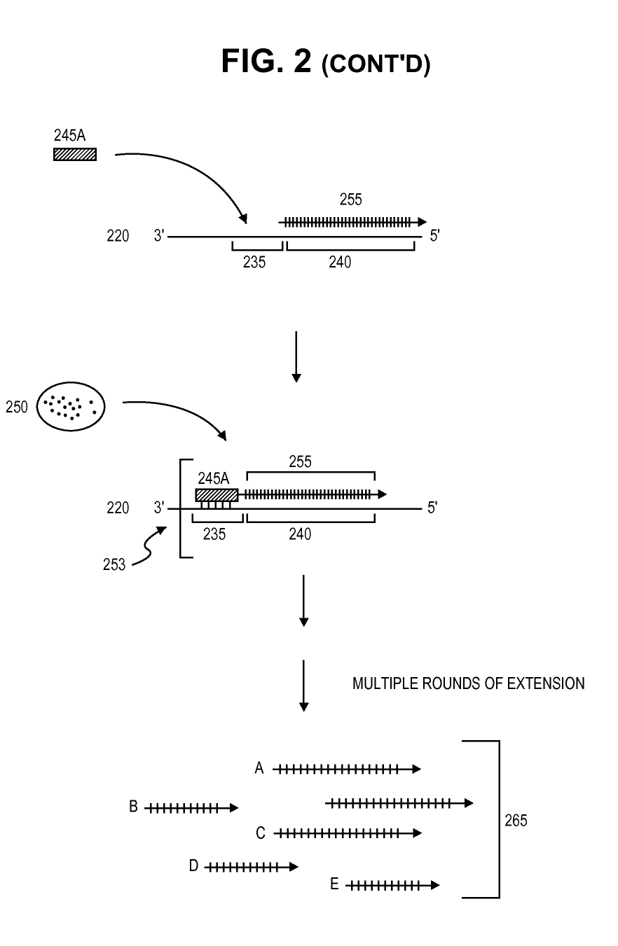

This example provides another exemplary method of single-strand polynucleotide amplification. The steps of this method are schematically depicted in FIG. 2.

Briefly, a single-stranded RNA 200 is provided. RNA 200 can be an mRNA extracted from a single cell sample or from a single cell. RNA 200 can be obtained from any source described herein using methods known to those of skill in the art. RNA 200 is then reverse transcribed to produce single-stranded cDNA 220, which comprises primer annealing site 235 and template sequence 240.

In a next step, RNA primer 245 hybridizes to primer annealing site 235 to form RNA/DNA hybrid 253. RNA primer 245 is then extended via the sequential addition of nucleotides in a template-specific manner by DNA polymerase 550 to produce target polynucleotide 255. The RNA portion RNA/DNA hybrid 253 is then cleaved (removed) by enzyme 260, which specifically digests RNA that is hybridized to DNA. The digestion of the RNA portion of RNA/DNA hybrid 253 by enzyme 260 exposes primer annealing site 235, permitting RNA primer 245A to hybridize to single-stranded cDNA 220. Primer 240A is then be extended by polymerase 250, displacing target polynucleotide 255 while producing another target polynucleotide.

Iterative rounds of RNA primer annealing, primer extension, and primer cleavage produces a population of single-stranded polynucleotides, which is represented in FIG. 2 as population 265.

1. A method of producing a population of single-stranded polynucleotides from a target polynucleotide, comprising:
(a) extending an RNA primer in a complex comprising:
(i) a DNA template comprising a sequence that is complementary to the target polynucleotide, and
(ii) the RNA primer, wherein the RNA primer is hybridized to the DNA template; and
(b) cleaving the RNA primer with an enzyme that cleaves RNA from an RNA/DNA hybrid such that another RNA primer hybridizes to the DNA template and repeats primer extension by strand replacement,
whereby a population of single-stranded polynucleotides is produced.

2. A method of analyzing a target polynucleotide, comprising:
(a) extending an RNA primer in a complex comprising:
(i) a DNA template comprising a sequence that is complementary to the target polynucleotide, and
(ii) the RNA primer, wherein the RNA primer is hybridized to the DNA template;
(b) cleaving the RNA primer with an enzyme that cleaves RNA from an RNA/DNA hybrid such that another RNA primer hybridizes to the DNA template and repeats primer extension by strand replacement, whereby a population of single-stranded polynucleotides are produced; and
(c) analyzing the single-stranded polynucleotides.

3. The method of claim 1, wherein the complex further comprises a termination polynucleotide sequence hybridized to a region on the DNA template that is 5′ to the region the RNA primer hybridizes to.

4. The method of claim 1, wherein the DNA template is produced from an RNA polynucleotide sequence.

5. The method of claim 1, wherein the DNA template is produced by:
(a) extending an RNA primer in a complex comprising:
(i) a DNA molecule and
(ii) an RNA primer, wherein the RNA primer is hybridized to the DNA molecule; and
(b) cleaving the RNA primer with an enzyme that cleaves RNA from an RNA/DNA hybrid such that another RNA primer hybridizes to the DNA template and repeats primer extension by strand replacement, whereby the DNA template is produced.

6. A method of producing a population of single-stranded polynucleotides from a target polynucleotide sequence comprising incubating a reaction mixture, the reaction mixture comprising:
(a) a DNA template comprising a sequence that is complementary to the target polynucleotide;
(b) an RNA primer hybridizable to the DNA template,
(c) a DNA polymerase, and
(d) an enzyme that cleaves RNA from an RNA/DNA hybrid,
wherein the incubation is under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced.

7. A method of analyzing a target polynucleotide; comprising:
1) producing a population of single-stranded polynucleotides generated by a method comprising incubating a reaction mixture comprising:
(a) a DNA template comprising a sequence that is complementary to the target polynucleotide;
(b) an RNA primer hybridizable to the DNA template,
(c) a DNA polymerase, and
(d) an enzyme that cleaves RNA from an RNA/DNA hybrid,
wherein the incubation is performed under a condition that permits primer hybridization, primer extension, RNA cleavage, and displacement of the primer extension product from the template when RNA is cleaved from the primer extension product whereby another RNA primer hybridizes and repeats primer extension by strand displacement, whereby multiple copies of single-stranded polynucleotides are produced; and
2) analyzing the single-stranded polynucleotides.

8. The method of claim 1, wherein the RNA primer is about 6 to about 20 nucleotides long.

9. The method of claim 1, wherein the RNA primer comprises a polyA sequence.

10. The method of claim 1, wherein the RNA primer comprises a random primer sequence.

11. The method of claim 1, wherein the DNA template comprises an adaptor sequence, and the RNA primer comprises a sequence that hybridizes to the adaptor sequence.

12. The method of claim 1, wherein the extension is carried out by a DNA polymerase selected from a group consisting of a strand displacing DNA polymerase, a high-fidelity DNA polymerase, a polymerase that has proofreading activity, a T7 DNA polymerase, and an E. coli DNA polymerase I.

13. The method of claim 1, wherein the enzyme that cleaves RNA from the RNA/DNA hybrid is RNase H.

14. The method of claim 1, wherein the DNA template is genomic DNA.

15. A kit for use in single-strand polynucleotide amplification, comprising: a) an RNA primer; b) a DNA polymerase, and c) an enzyme that cleaves RNA from an RNA/DNA hybrid.

16. The kit of claim 15, wherein the enzyme that cleaves RNA from the RNA/DNA hybrid is RNase H.

17. The kit of claim 15, wherein the DNA polymerase is selected from the group consisting of selected from a group consisting of a strand displacing DNA polymerase, a high-fidelity DNA polymerase, a polymerase that has proofreading activity, a T7 DNA polymerase, and an E. coli DNA polymerase I.

18. The kit of claim 15, wherein the RNA primer is about 6 to about 20 nucleotides long.

19. The kit of claim 15, wherein the RNA primer comprises a polyA sequence.

20. The kit of claim 15, wherein the RNA primer comprises a random primer sequence.
